(19) United States
(12) Patent Application Publication
(10) Pub. No.: US 2025/0000386 A1
(43) Pub. Date: Jan. 2, 2025

(54) CAPNOGRAPHY-BASED EVALUATION OF RESPIRATORY OBSTRUCTION LEVEL

(71) Applicant: Oridion Medical 1987 Ltd., Jerusalem (IL)

(72) Inventors: Michal Ronen, Givat Brenner (IL); Yaniv Refaelovich, Givatayim (IL); Yoni Schwartz, Jerusalem (IL)

(21) Appl. No.: 18/710,556

(22) PCT Filed: Nov. 17, 2022
(86) PCT No.: PCT/IL2022/051229

§ 371 Date: May 15, 2024

Related U.S. Application Data
(60) Provisional application No. 63/281,144, filed on Nov. 19, 2021.

(51) Int. Cl. A61B 5/083; A61B 5/00; G16H 10/60; G16H 50/30

(52) U.S. Cl. CPC A61B 5/0836; A61B 5/7239; A61B 5/7267; A61B 5/7282; G16H 10/60; G16H 50/30

(57) ABSTRACT

Disclosed are computer-implemented methods and related systems for evaluating a respiratory obstruction-level in a subject with a respiratory condition, based on a capnograph signal of the subject. A first plurality of waveform features, derived from the capnograph signal, is used to discard invalid breath signals, thereby pre-processing the capnograph signal. A second plurality of waveform features, derived from the pre-processed signal, is fed into a machine learning algorithm to obtain a score quantifying the respiratory obstruction level of the subject.

### TECHNICAL FIELD

The present disclosure relates generally to evaluation of a respiratory obstruction level in subjects with a respiratory condition.

### BACKGROUND

A capnograph is a medical device that measures CO2 concentration in exhaled breath of a subject. The measurement results are typically presented as a capnogram: a plot of expiratory CO2 (measured in millimeters of mercury, mmHg) as a function of time or expired volume. The shape of the plot may be referred to as the “waveform”.

### SUMMARY

Aspects of the disclosure, according to some embodiments thereof, relate to evaluation of a respiratory obstruction-level in subjects (e.g. patients) with a respiratory condition. More specifically, but not exclusively, aspects of the disclosure, according to some embodiments thereof, relate to evaluation of a respiratory obstruction-level in subjects with a respiratory condition based on capnography data.

Currently, spirometry is considered the gold standard for estimating respiratory obstruction levels. Even though a spirometer is a non-invasive and simple instrument, it requires effort and full cooperation on the part of the subject. Thus, in uncooperative populations or during medical-emergencies, the use of spirometry may be problematic. The present disclosure, according to some embodiments thereof, addresses the above-mentioned problem by providing methods for estimating a respiratory obstruction-level of a subject using capnography. In contrast to spirometry, capnography requires no effort and little cooperation from the subject. Advantageously, according to some embodiments of the disclosed methods, the use of capnography does not come at the expense of precision. That is, the disclosed methods allow for obstruction-level estimates that are at least as accurate as state-of-the-art estimates obtained using spirometry.

Another advantage of the disclosed methods is the provision of an obstruction-level measure which is substantially continuous (being able to distinguish between at least 100 obstruction levels). Further, the estimate may be provided substantially continuously, that is, updated in real-time after every (single) breath of the subject. Advantageously, due to the substantially continuous character thereof, both in amplitude and in time, the measure can be used to monitor fluctuations in the obstruction-level and in the response to a treatment, and thereby potentially improve therapy. Moreover, the disclosed methods allow for daily monitoring at home.

Thus, according to an aspect of some embodiments, there is provided a computer-implemented method for evaluating a respiratory obstruction-level in a subject with a respiratory condition. The method includes:
obtaining a capnograph signal of a subject;
pre-processing the capnograph signal by:
   segmenting the capnograph signal into single-breath signals;
   extracting a first plurality of waveform features from the capnograph signal;
   based on the first plurality of waveform features, discarding from the capnograph signal invalid single-breath signal(s);
evaluating a respiratory obstruction-level of the subject by:
   extracting a second plurality of waveform features derived from the pre-processed signal;
   feeding the second plurality of waveform features into a machine learning algorithm (MLA) to obtain a score quantifying the respiratory obstruction level of the subject.

According to some embodiments, the respiratory condition is, or results from, asthma, chronic obstructive pulmonary disease (COPD), cystic fibrosis (CF), and/or a lung tumor(s).

According to some embodiments, the MLA is an artificial neural network (ANN), a convolutional neural network, a random forest model, a fuzzy logic, a Bayesian network, a decision tree, a radial base function, a support vector machine, a linear regression model, a non-linear regression model, an expert system, or any combination thereof.

According to some embodiments, inputs of the MLA include an input specifying the respiratory condition.

According to some embodiments, the score allows distinguishing between at least 100 different respiratory obstruction levels.

According to some embodiments, the score is substantially continuous.

According to some embodiments, in the step of pre-processing the capnograph signal, the discarding of invalid single-breath signals is implemented using an auxiliary MLA.

According to some embodiments, the second plurality of waveform features includes multi-breath waveform features.

According to some embodiments, the second plurality of waveform features includes at least 20 waveform features.

According to some embodiments, the second plurality of waveform features includes the waveform features of Table 2, and/or any functions thereof.

According to some embodiments, the second plurality of waveform features includes at least 17 of the waveform features of Table 3, and/or any functions thereof.

According to some embodiments, in addition to the second plurality of waveform features, inputs of the MLA include demographic data characterizing the subject.

According to some embodiments, the demographic data includes at least one of gender, age, height, ethnicity, and weight of the subject.

According to some embodiments, in addition to the second plurality of waveform features, inputs of the MLA include treatment data including one or more of the following treatment parameters: a binary parameter specifying administration or no administration of O₂, rate of O₂ administration, and/or a binary parameter specifying provision or no provision of an inhaler.

According to some embodiments, the first plurality of waveform features includes at least 5 waveform features.

According to some embodiments, the first plurality of waveform features includes at least 5 of the waveform features of Table 1, and/or any functions thereof.

According to some embodiments, the MLA is an ANN.

According to some embodiments, weights and/or architecture of the ANN are dependent on the respiratory condition.

According to some embodiments, the auxiliary MLA is an auxiliary ANN.

According to an aspect of some embodiments, there is provided a computer-readable storage medium including software executable by a computer processor(s) for evaluating a respiratory obstruction-level in a subject with a respiratory condition. The software is configured, given a capnograph signal of a subject as an input, to implement steps of pre-processing the capnograph signal and evaluating a respiratory obstruction-level of the subject as described above.

According to an aspect of some embodiments, there is provided a capnograph including a computer processer(s) and a computer-readable storage medium as described above. The capnograph is thereby configured to implement the method(s) described above.

According to an aspect of some embodiments, there is provided a computer-implemented method for evaluating a respiratory obstruction-level in a subject with a respiratory condition. The method includes the steps of:
Obtaining a capnograph signal of a subject including at least one single-breath signal;
pre-processing the capnograph signal by
   identifying a single-breath signal in the capnograph signal;
   extracting a first plurality of waveform features from the single-breath signal;
   determining whether the single-breath signal is invalid based on the first plurality of waveform features; and
   if so, discarding the single-breath signal, else
evaluating a respiratory obstruction-level of the subject by
   extracting a second plurality of waveform features derived from the pre-processed signal;
   feeding the waveform features in the second plurality of waveform features into a machine learning algorithm (MLA) to obtain a score quantifying the respiratory obstruction level of the subject.

According to some embodiments, the MLA is an artificial neural network (ANN), a convolutional neural network, a random forest model, a fuzzy logic, a Bayesian network, a decision tree, a radial base function, a support vector machine, a linear regression model, a non-linear regression model, an expert system, or any combination thereof.

According to some embodiments, the second plurality of waveform features consists of waveform features extracted from the single-breath signal.

According to some embodiments, wherein the second plurality of waveform features includes waveform features extracted from multi-breath signals, the multi-breath signals include the single-breath signal.

According to some embodiments, the multi-breath signals include at least two single-breath signals.

According to some embodiments, the method is effected repeatedly for consecutive single-breath signals of the subject.

According to some embodiments, the method is effected in real-time as the subject is monitored by a capnograph, which capnograph is used to obtain the capnograph signal(s).

According to some embodiments, in each repetition the identified single-breath signal is a last-obtained single-breath signal.

According to some embodiments, the capnograph signal consists of the identified signal-breath signal.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

Embodiments of the present disclosure may include apparatuses for performing the operations herein. The apparatuses may be specially constructed for the desired purposes or may include a general-purpose computer(s) selectively activated or reconfigured by a computer program stored in the computer. Such a computer program may be stored in a computer readable storage medium, such as, but not limited to, any type of disk including floppy disks, optical disks, CD-ROMs, magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions, and capable of being coupled to a computer system bus. It is noted that embodiments of the present disclosure may be cloud-based or involve cloud computing.

The processes and displays presented herein are not inherently related to any particular computer or other apparatus. Various general-purpose systems may be used with programs in accordance with the teachings herein, or it may prove convenient to construct a more specialized apparatus to perform the desired method(s). The desired structure(s) for a variety of these systems appear from the description below. In addition, embodiments of the present disclosure are not described with reference to any particular programming language. It will be appreciated that a variety of programming languages may be used to implement the teachings of the present disclosure as described herein.

Aspects of the disclosure may be described in the general context of computer-executable instructions, such as program modules, being executed by a computer. Generally, program modules include routines, programs, objects, components, data structures, and so forth, which perform particular tasks or implement particular abstract data types. Disclosed embodiments may also be practiced in distributed computing environments where tasks are performed by remote processing devices that are linked through a communications network. In a distributed computing environment, program modules may be located in both local and remote computer storage media including memory storage devices.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the figures:

FIG. 1 schematically depicts phases in a typical capnography waveform corresponding to a single-breath signal;

FIG. 2 is a flowchart of a capnography-based method for evaluating a respiratory obstruction-level in a subject with a respiratory condition, according to some embodiments;

FIGS. 3A-3F schematically depict valid single-breath waveforms (FIGS. 3A-3C) and invalid single-breath waveforms (FIGS. 3D-3F);

FIG. 4 presents a scatter plot comparing respiratory obstruction-level estimates from a plurality of subjects obtained (i) using a capnograph and applying the disclosed methods to process the capnograph signal, and (ii) using a spirometer, according to some embodiments;

FIG. 5 presents a normalized score quantifying the respiratory obstruction-level of a subject as a function of time, the normalized score having been obtained using the disclosed capnography-based methods, according to some embodiments; mean normalized scores, each averaged over a respective 5-minute interval, and % FEV1s, obtained using a spirometer following each of the 5-minute intervals, are also presented;

FIGS. 6A and 6B present the effects of O₂ administration on the normalized scores, obtained using the disclosed capnography-based methods, according to some embodiments, on two different subjects, respectively; the normalized score is shown as a function of time;

FIGS. 7A and 7B are statistical error curves illustrating the effects of discounting and taking into account demographic data, respectively, in the implementation of the disclosed capnography-based methods, according to some embodiments; and

FIG. 8 is a receiver operating characteristic (ROC) curve derived from outputs of an auxiliary ANN for classifying validity of capnography signals, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the description and claims of the application, the words “include” and “have”, and forms thereof, are not limited to members in a list with which the words may be associated.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

As used herein, according to some embodiments, the term “waveform”, with reference to a capnograph obtained signal, and the term “capnogram” may be interchangeable.

As used herein, % FEV1 is defined as the ratio of the measured FEV1 (forced expiratory volume in 1 second) to predicted FEV1 (which depends on the gender, age, height, weight, and ethnicity of the subject).

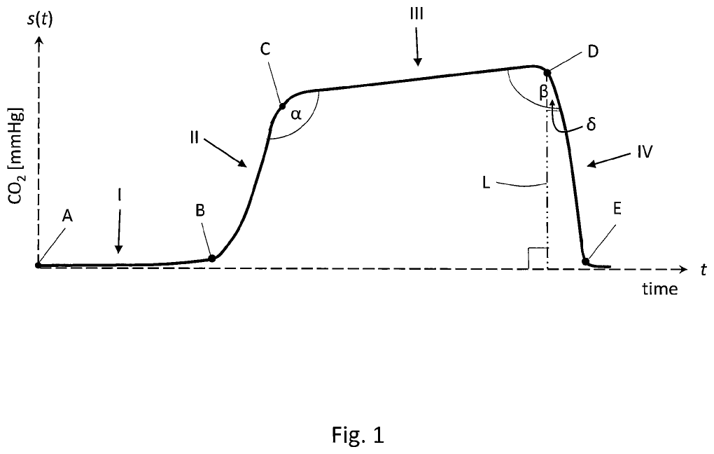

FIG. 1 schematically depicts a capnogram of a (valid) single-breath of a subject. The concentration of CO₂ in the exhaled breath (i.e. the signal s) is shown plotted as a function of time t and exhibits a (valid) waveform. The waveform includes four successive phases I-IV. Phase I, from a point A=(Aₜ, Aₛ) (i.e. the point characterized by the coordinates t=Aₜ and s=Aₛ) to a point B=(Bₜ, Bₛ), corresponds to the inspiratory baseline, wherein the subject inhales so that substantially no CO₂ is detected by the capnograph. Phase II, from point B to a point C=(Cₜ, Cₛ), corresponds to the expiratory upstroke, wherein the subject begins exhaling. The concentration of CO₂ increases with time as the composition of the exhaled breath increasingly shifts from being dominated by gas, originating in dead space of the airways, to CO₂ rich gas from the alveoli. Point B marks the beginning of the expiratory upstroke. Phase III, from point C to a point D=(Dₜ, Dₛ), corresponds to the alveolar plateau, wherein the exhaled breath is substantially CO₂ rich gas from the alveoli. Point C marks the beginning of the alveolar plateau. Phase IV, from point D to a point E=(Eₜ, Eₛ) corresponds to the inspiratory downstroke, wherein the subject begins inhaling, and accordingly the CO₂ concentration rapidly decreases. Point D marks the beginning of the inspiratory downstroke and point E marks the end of the inspiratory downstroke (and the end of the (single) breath and the beginning of the next breath, i.e. the beginning of the inspiratory baseline of the next breath of the subject). An angle α indicates the angle between the curve of phase II and the curve of phase III. An angle β indicates the angle between the curve of phase III and the curve of phase IV. An angle δ indicates the angle between a vertical (straight) line L, passing through point D, and the curve of phase IV. According to some embodiments, Aₜ=0, i.e. the signal commences at t=0.

### Methods

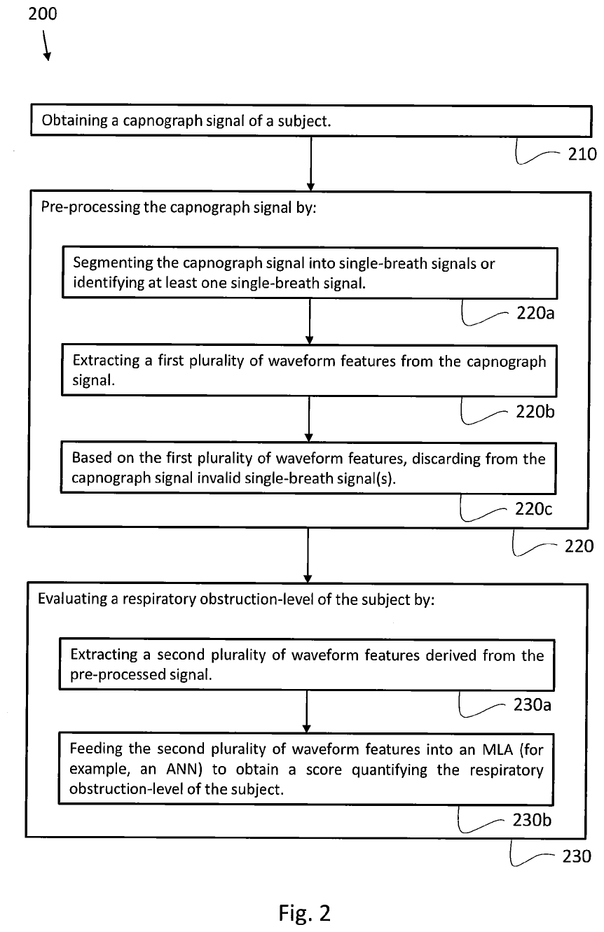

FIG. 2 is a flowchart of a computer-implemented method 200 to detect and quantify a respiratory obstruction level of a subject with a respiratory condition, based on capnography-obtained data of the subject, according to some embodiments. According to some embodiments, the respiratory condition is, or results from, asthma, chronic obstructive pulmonary disease (COPD), cystic fibrosis (CF), and/or a lung tumor(s).

According to some embodiments, method 200 includes:
A step 210 wherein a capnograph signal of the subject is obtained.
A step 220 wherein the capnograph signal is pre-processed. Step 220 includes the sub-steps of:
   A sub-step 220a wherein the capnograph signal is segmented into single-breath signals or wherein at least one single-breath signal is identified.
   A sub-step 220b wherein, for each single-breath signal, a first plurality of waveform features is extracted from the capnograph signal.
   A sub-step 220c wherein, based on the first plurality of waveform features, the single-breath signals are classified into valid and invalid breath signals, and invalid breath signals are discarded to obtain a pre-processed signal.
A step 230 wherein the respiratory obstruction-level of the subject is evaluated using a machine learning algorithm (MLA), for example, an artificial neural network. Step 230 includes the sub-steps of:
   A sub-step 230a wherein a second plurality of waveform features is extracted from the pre-processed signal.
   A sub-step 230b wherein, the second plurality of waveform features, and optionally demographic data characterizing the subject, are fed into the MLA to obtain a score quantifying the respiratory obstruction-level.

According to some embodiments, the capnograph signal, obtained in step 210, is a multi-breath signal (i.e. including a plurality of single-breath signals). According to some such embodiments, the second plurality of waveform features may include multi-breath waveform features including information pertaining to two or more single-breaths, such as averages and correlations between single-breath waveform features. More specifically, as used herein, multi-breath waveform features may refer to (i) “non-intrinsically” multi-breath waveform features, which constitute feature averages (wherein a “feature average” is obtained by averaging the values of a waveform feature, each value obtained from its corresponding single-breath signal, wherein a plurality of single-breath signals make up the multi-breath signal), and (ii) “intrinsically” multi-breath waveform features, which cannot be derived by taking a feature average (as defined above). Examples of intrinsically multi-breath waveform features include the highest-power frequency in the signal, the ratio between the total amount of time the signal rises and the total amount of time the signal falls (over many breaths), and the Hjorth mobility of the signal. As used herein, according to some embodiments, the term “signal” may refer to a concatenation of two or more consecutive single-breaths signals.

According to some embodiments, the capnograph signal is composed of at least 2 consecutive single-breath signals (corresponding to 2 successive inhalation-exhalation cycles of the subject), at least 5 consecutive single-breath signals, at least 10 consecutive single-breath signals, or even at least 20 consecutive single-breath signals. Each possibility is a separate embodiment. According to some embodiments, the obtained capnograph signal is obtained from a continuous measurement over at least about 12 seconds, at least about 20 seconds, at least about 30 seconds, at least about 1 minute, or even at least about 2 minutes. Each possibility is a separate embodiment.

According to some embodiments, the capnograph signal is a single-breath signal.

Table 1 lists the first plurality of waveform features extracted, in sub-step 220b, for each of the single-breath signals (obtained in sub-step 220a by the segmentation of the capnograph signal), according to some embodiments. The waveform features in the first plurality may be computed for each of the single-breath signals. The term “normalized signal” in Table 1, with reference to a single-breath signal, refers to the single-breath signal after having undergone normalization (linear rescaling), such that the maximum of the normalized single-breath signal equals 1 and the minimum equals 0.

According to some embodiments, the first plurality of waveform features includes the waveform features of Table 1, and/or any functions thereof. According to some embodiments, the first plurality of waveform features includes 5 or more of the waveform features of Table 1, and/or any functions thereof.

TABLE 1
The first plurality of waveform features,
according to some embodiments.
Waveform feature

| | |
|---|---|
| 1. | Area under the signal's (e.g. s in FIG. 1) second derivative squared |
| 2. | Number of times the signal's derivative crosses the time axis |
| 3. | The absolute difference between the highest peak and the lowest |
| | peak in the signal between times Cₜ and Dₜ |
| | (the time coordinates of points C and D, respectively, |
| | wherein t = 0 marks the beginning of the signal) |
| 4. | Normalized signal at 0.2 · Dₜ (i.e. the value |
| | of the normalized signal at the time t = 0.2 × Dₜ) |
| 5. | Normalized signal at 0.3 · Dₜ |
| 6. | Normalized signal at 0.5 · Dₜ |
| 7. | Normalized signal at 0.7 · Dₜ |
| 8. | Normalized signal at 0.8 · Dₜ |

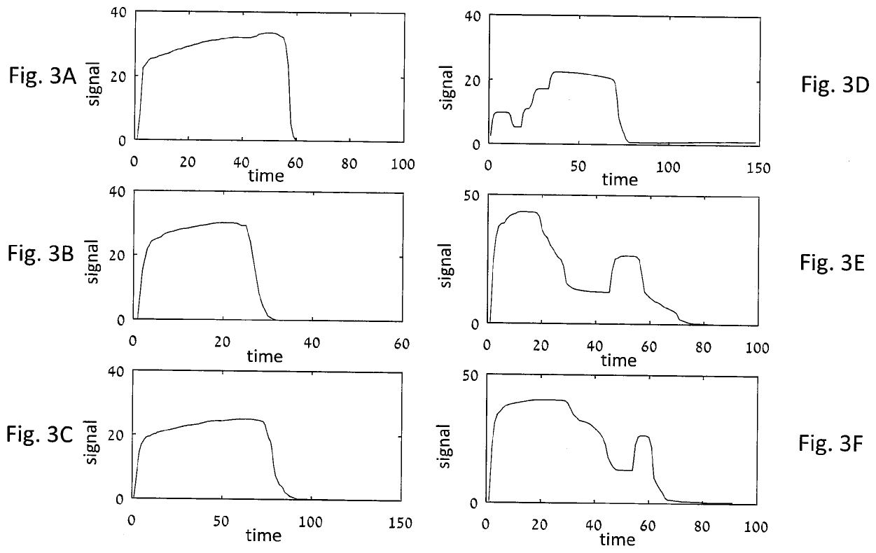

In sub-step 220c, each of the single-breath signals may be classified as valid or invalid. Invalid breath signals may be characterized by waveforms having abnormal and/or distorted shapes. According to some embodiments, an invalid breath signal may be a signal which contains substantially no information or lacks a sufficient amount of information regarding the respiratory status of the subject due to, for example, noise or incorrect placement of the capnograph cannula or mask. FIGS. 3A-3F depict exemplary waveforms of valid single-breath signals (FIGS. 3A-3C) and invalid single-breath signals (FIGS. 3D-3F). Invalid single-breath signals are discarded to obtain the pre-processed signal, which is then analyzed in step 230 to estimate the obstruction-level of the subject.

According to some embodiments, in sub-step 220c, the classification of the single-breath signals into valid and invalid breath signals is effected using an auxiliary (i.e. additional) MLA (different from the MLA used to obtain the obstruction-level in sub-step 230b). That is, each of the waveform features (from the first plurality) of a single-breath signal may be fed as a separate input into the auxiliary MLA, and the output of the auxiliary MLA may be indicative of whether the single-breath signal is valid or invalid (e.g. the output may be binary).

Table 2 lists the second plurality of waveform features, extracted in sub-step 230a, according to some embodiments. Table 3 lists the second plurality of waveform features, extracted in sub-step 230a, according to some embodiments. Tables 2 and 3 correspond to separate embodiments, respectively.

TABLE 2
The second plurality of waveform features,
according to some embodiments.
Waveform feature

| | |
|---|---|
| 1. | Total amount of time signal rises |
| 2. | Ratio of the total amount of time signal |
| | rises to the total amount of time signal falls |
| 3. | Hjorth mobility of signal |
| 4. | Average slope of linear regression fit of the |
| | signal computed from data points between Cₜ and Dₜ |
| 5. | Average of the ratio of the time until Dₜ to the |
| | time from Dₜ to the end of the signal (i.e. Dₜ/(Eₜ − Dₜ)) |
| 6. | Average R² of linear regression fit computed |
| | from data points between t = 0 and Dₜ |
| 7. | Average mean-square error (MSE) of exponential fit of signal |
| | (the exponential fit being computed for each single breath signal) |
| 8. | Average normalized signal at 0.5 · Dₜ |
| 9. | Average time to signal peak |
| 10. | Average time to signal peak divided by Dₜ |
| 11. | Average slope between points C and D |
| 12. | Average time spent in EtCO₂ (end tidal CO₂) |
| 13. | Average angle of the downward slope (δ) |
| 14. | Average distance from point C to the |
| | straight line connecting (0, 0) to point D |
| 15. | Average ratio of the two areas defined by the |
| | signal from the middle thereof (i.e. at Eₜ/2) |
| | to the end thereof and from the start thereof to the middle |
| 16. | Average difference between the length of the signal curve from (0, 0) |
| | to point D and the length of the straight line from (0, 0) to point D |
| 17. | Average curvature of signal from start to point D |

It is noted that the averaging referred to in Tables 2 and 3, e.g. the average normalized signal at 0.5. Dₜ, refers to the feature average (as computed from values of the (waveform) feature pertaining to each valid single-breath signal in the multi-breath signal), i.e. the (waveform) feature average of a quantity x is given by

$$〈 x 〉 = \frac{1}{n} {Σ}_{i = 1}^{n} {x}_{i} ,$$

wherein n is the number of (valid) single-breaths signals making up the signal and xᵢ is the value the quantity assumes in the i-th single-breath signal. Thus, for example, item 8 in Table 2, the average normalized signal at 0.5·Dₜ, in more detail could have been expressed as

$$\frac{1}{2 n} {Σ}_{i = 1}^{n} {D}_{t}^{( i )} ,$$

wherein Dₜ⁽ⁱ⁾ is the time coordinate of the point D⁽ⁱ⁾ which marks the beginning of the downstroke of the i-th (single) breath, and wherein Dₜ⁽ⁱ⁾ is understood to be computed from the beginning of the i-th breath signal (e.g. for the purposes of the averaging, Aₜ⁽ⁱ⁾ is set to equal 0, wherein point A⁽ⁱ⁾ marks the beginning of the i-th breath signal).

According to some embodiments, the second plurality of waveform features includes the waveform features of Table 2, and/or any functions thereof. According to some embodiments, the second plurality of waveform features includes the waveform features of Table 3, and/or any functions thereof. According to some embodiments, the second plurality of waveform features includes at least 10, at least 15, or at least 20, of the waveform features of Table 3, and/or any functions thereof. Each possibility is a separate embodiment.

TABLE 3
The second plurality of waveform features,
according to some embodiments.
Waveform feature

| | |
|---|---|
| 1. | Total amount of time signal rises |
| 2. | Ratio of the total amount of time signal |
| | rises to the total amount of time signal falls |
| 3. | Hjorth mobility of signal |
| 4. | Average of the ratio of the slopes between points |
| | C and D and between (0, 0) and point C |
| 5. | Average slope of linear regression fit of the |
| | signal computed from data points between Cₜ and Dₜ |
| 6. | Average of the ratio of the time until Dₜ |
| | to the time from Dₜ to the end of the signal |
| 7. | Average of the ratio of the amount of time the signal |
| | rises to the amount of time the signal falls, computed Cₜ to end |
| 8. | Average R² of linear regression fit computed |
| | from data points between t = 0 and Dₜ |
| 9. | Average mean-square error (MSE) of exponential fit of signal |
| | (the exponential fit being computed for each single breath signal) |
| 10. | Average of mean of signal between start and Dₜ |
| 11. | Average normalized signal at 0.3 · Dₜ |
| 12. | Average normalized signal at 0.4 · Dₜ |
| 13. | Average normalized signal at 0.5 · Dₜ |
| 14. | Average normalized signal at 0.6 · Dₜ |
| 15. | Average normalized signal at Cₜ |
| 16. | Average time to signal peak |
| 17. | Average time to signal peak divided by Dₜ |
| 18. | Average of signal at the point where the straight |
| | line running through (0, 1) and (1, 0) crosses the signal |
| 19. | Average of signal at the point where the straight line running through |
| | (0, 1) and (1, 0) crosses the signal divided by Dₛ |
| | (i.e. the signal is attenuated by 1/Dₛ) |
| 20. | Average slope between points C and D |
| 21. | Average time spent in EtCO₂ |
| 22. | Average normalized signal at 0.35 · Dₜ |
| 23. | Average normalized signal at 0.45 · Dₜ |
| 24. | Average normalized signal at 0.55 · Dₜ |
| 25. | Average angle of the downward slope (see δ in FIG. 1) |
| 26. | Average normalized signal at Dₜ/2 |
| 27. | Average of b, wherein b is the intercept of linear regression |
| | fit of the signal computed for data points between Cₜ and Dₜ |
| 28. | Average distance from point C to the |
| | straight line connecting (0, 0) to point D |
| 29. | Average ratio of the two areas defined by the signal from |
| | the middle thereof (i.e. at Eₜ/2) to the end |
| | thereof and from the start thereof to the middle |
| 30. | Average difference between the length of the signal curve from (0, 0) |
| | to point D and the length of the straight line from (0, 0) to point D |
| 31. | Average curvature of signal from start to point D |
| 32. | Average curvature of signal from start to |
| | the time coordinate midway between Cₜ and Dₜ |

According to some embodiments, the second plurality of waveform features may additionally include non-intrinsically multi-breath waveform features equivalents of intrinsically multi-breath waveform features. For example, referring to item 2 in Table 2, according to some embodiments, the second plurality of waveform features may additionally/alternatively include the average of the ratio of the amount of time a (single-breath) signal rises to the amount of time signal falls.

According to some embodiments, one or more of the averaged quantities/parameters in Table 2 and/or Table 3 may be replaced by the median thereof, the standard deviation thereof and/or other suitable statistical parameters thereof. According to some embodiments, when the second plurality of waveform features includes one or more quantities/parameters that have been averaged over, the second plurality of waveform features may additionally or alternatively include the median, the standard deviation, or other suitable statistical parameter of at least one of the one or more quantities/parameters. As a non-limiting example, according to some embodiments, the second plurality of waveform features may further or alternatively include Table 2, the medians and/or standard deviations of all quantities/parameters that appear as averages in Table 2.

It will be understood that Table 2 and Table 3 can also be computed in the case that the capnograph signal is a single-breath signal or in the case that only a single valid breath signal is identified. In such cases, averaged quantities/parameters do not have to be averaged (i.e. feature averages do not have to be taken, as the waveform feature “average” is just the value of the waveform feature in the identified single valid breath signal).

According to some embodiments, the MLA used to implement sub-step 230b is an artificial neural network (ANN). According to some embodiments, the MLA may be a convolutional neural network, a random forest model, a fuzzy logic, a Bayesian network, a decision tree, a radial base function, a support vector machine, a linear regression model, a non-linear regression model, an expert system, or any combination thereof, as well as any combination thereof with an ANN(s).

According to some embodiments, wherein the classification of the single-breath signals into valid and invalid breath signals in sub-step 220c is effected using the auxiliary MLA, the auxiliary MLA is an auxiliary ANN. According to some embodiments, the auxiliary MLA may be a convolutional neural network, a random forest model, a fuzzy logic, a Bayesian network, a decision tree, a radial base function, a support vector machine, a linear regression model, a non-linear regression model, an expert system, or any combination thereof, as well as any combination thereof with an ANN(s).

According to some embodiments, in sub-step 230b, the score is computed taking into account demographic data characterizing the subject. According to some embodiments, the demographic data includes one or more of the gender, age, height, ethnicity, and/or weight of the subject. According to some embodiments, the demographic data includes at least the gender, age, and height of the subject.

According to some embodiments, each of the waveform features (from the second plurality), and optionally each datum from the demographic data, may be fed as a separate input into the MLA (used to perform sub-step 230b). The output of the MLA may be indicative of the obstruction-level. According to some embodiments, the output is the score quantifying the obstruction-level. According to some embodiments, the output may allow distinguishing between at least 100 different obstructions levels. According to some embodiments, the output (and the score) are effectively or substantially continuous, and the score may range from 0 to 100. As used herein, the term “normalized score” refers to the score when scaled such as to range from 0 to 1.

According to some embodiments, weights and/or architecture of the MLA may be dependent on the specific respiratory condition. That is, according to some embodiments, the weights, the number of hidden layers, or even the number of inputs and choice of inputs (e.g. the choice of waveform features) may be dependent on the respiratory condition (e.g. asthma or COPD). According to some embodiments, the MLA includes an input specifying the respiratory condition.

According to some embodiments, the MLA includes inputs specifying treatment data, such as whether the subject (e.g. patient) is administered oxygen (O₂) or not, the rate of oxygen administration, whether or not the subject is provided with an inhaler, whether the subject is ventilated (e.g. intubated), and so on, as known in the art of treatment of respiratory conditions and capnography.

According to some embodiments, the MLA includes an input specifying the position of the subject when the capnograph signal is obtained therefrom, i.e. whether the subject is standing, sitting, or supine.

According to some embodiments, following the computation of the score in sub-step 230b, steps 210-230 may be repeated to obtain an updated score. According to some such embodiments, in sub-step 230a multi-breath waveform features characterizing single-breath signals obtained in the present repetition and in the previous repetition(s) may additionally be extracted. As a non-limiting example, such a multi-breath waveform feature may correspond to the average of a waveform feature of the first obtained single-breath signal in the present repetition and a (same) waveform feature of the last obtained single-breath signal in the previous repetition. According to some such embodiments, wherein in each repetition a single-breath signal is obtained (i.e. in step 210 a single-breath signal is measured), or wherein in step 210 the capnograph signal is obtained over a timeframe of about 3 sec to about 5 sec (corresponding to the average duration of a single inhalation-exhalation cycle of an adult), the score is accordingly updated (e.g. every 3 sec when the timeframe is 3 sec), and thereby generated in a substantially continuous or near continuous manner. That is, steps 210-230 may be repeated such as to effect a moving-window analysis. According to some such embodiments, the moving window captures at least 2 last (single) breaths of the subject, at least 5 last breaths of the subject, at least 10 last breaths of the subject, or even at least 20 last breaths of the subject. Each possibility is a separate embodiment.

### Clinical Study Results

This sub-section presents results demonstrating the feasibility and efficacy of the disclosed methods, according to some embodiments thereof. Data was collected during two clinical studies, held at the pulmonary clinic at the Rabin Medical Center in Petah Tikva, Israel. The first study included both healthy and moderately asthmatic subjects who underwent a methacholine challenge test for asthma diagnosis. The second study included both asthmatic subjects, and subjects with COPD, with more severe respiratory conditions (as compared to the subjects in the first study), who attended a routine check. All subjects were over the age of twenty. In both studies, capnography waveforms were continuously recorded, and then analyzed on a computer using the disclosed methods. More specifically, in the second study, the capnography measurements were divided into 5-minute intervals, with each followed by a spirometry test before resuming the capnography measurement in the next time-interval, while in the first study, each capnography interval was preceded by a spirometry test.

Each of the subjects also underwent spirometry tests to determine their % FEV1 (and thereby estimate their respiratory obstruction-levels independently of the disclosed capnography-based methods). The (spirometry-obtained) % FEV1 were later used as a reference to evaluate the estimation accuracy of the disclosed (capnography-based) methods.

Capnography measurements were recorded using a Smart CapnoLine® connected to a Capnostream 20p device. The spirometry tests were performed by a certified technician using a spirometry device.

Following pre-processing to discard invalid single-breath signals, the obtained data was analyzed using an ANN with 35 inputs corresponding to the 32 waveform features of Table 3, and the demographic features of gender, age, and height. The ANN included one hidden layer with 23 nodes and was further characterized by a sigmoid-activation. Each sequence of computed (obtained in a respective 5-minute interval) scores was averaged to obtain a mean score.

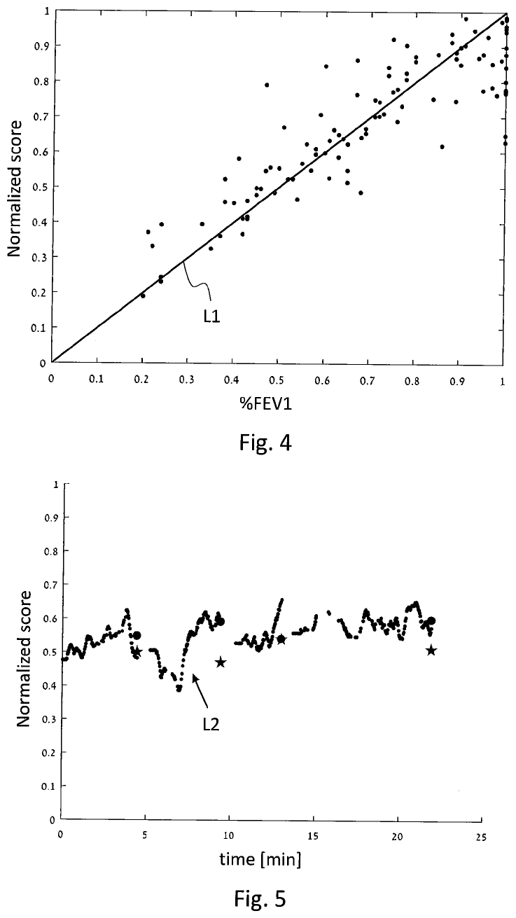

FIG. 4 is a scatter plot displaying the mean (normalized) scores (quantified by the y (i.e. vertical) axis), and % FEVIs (quantified by the x (i.e. horizontal) axis) obtained using the spirometer. Each displayed point includes the mean normalized score as the y coordinate and the corresponding % FEV1 as the x coordinate. Each FEV1% measurement was performed immediately following the respective 5-minute interval.

To evaluate the agreement of the mean normalized scores and the % FEVIs, the distances of the points from the y=x curve (indicated by L1 in FIG. 4) were computed. Two statistical measures were calculated, showing good agreement between the two sets of data, with R-squared (R²) equaling 0.7 and the root-mean-square (RMSE) equaling 0.14.

FIG. 5 presents the normalized score of a single subject (from the second study) as a function of time. The normalized score is indicated by the plot L2. The stars correspond to the mean normalized score (averaged over the preceding 5 minutes). The dots correspond to the respective % FEVIs (as recorded by the spirometer).

The performance of the ANN was also investigated as dependent on the severity of the respiratory obstruction-level. The subjects were classified into 4 groups according to the level of respiratory obstruction (as determined from the % FEV1): mild obstruction level (% FEV1<0.3), moderate obstruction level (0.3<% FEV1<0.5), severe obstruction level (0.5<% FEV1<0.8), and very severe obstruction level (% FEV1<1). Table 4 presents the computed RMSE of each of the groups. The highest accuracy was achieved for the highest levels of obstruction.

TABLE 4
RMSE according to severity of the respiratory obstruction-level.

| Mild | Moderate | Severe | Very severe | All |
|---|---|---|---|---|
| 0.15 | 0.11 | 0.12 | 0.06 | 0.14 |

Various common treatment protocols for respiratory conditions include providing the subject with O₂. The O₂, which is administered via the cannula of the capnograph (into the subject's nose), may dilute the CO₂ and influence the measurement results. O₂ flow-rates of up to 5 liters per minute are approved by Medtronic and do not affect the CO₂ concentration measurement results or precision. Nevertheless, the presence of O₂ may potentially affect the shapes of the obtained waveforms and thereby affect the scores (outputs of the ANN).

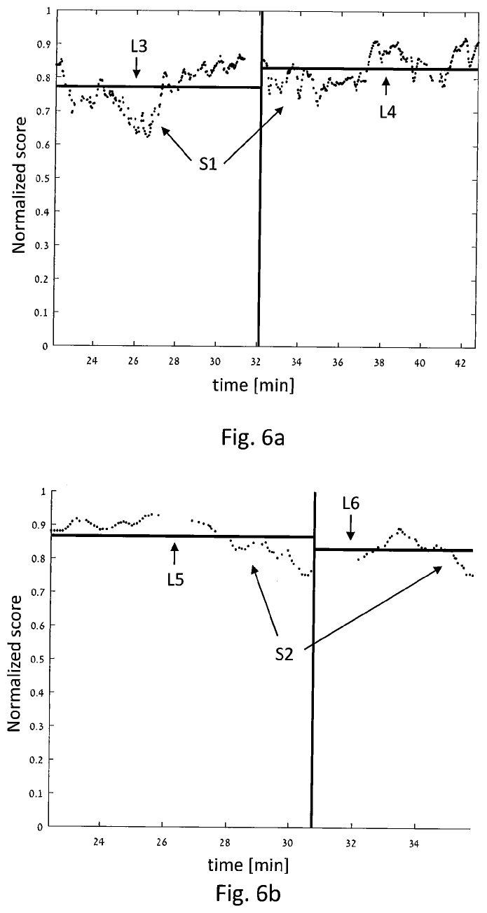

To investigate this possibility, in the second study, 13 of the subjects were administered O₂. More specifically, the 13 subjects were first monitored without receiving O₂, and later while receiving O₂. FIGS. 6A and 6B display the effects of O₂ administration on two different subjects, respectively. As shown in FIG. 6A, the effect of O₂ administration on the first subject translated to an increase in the mean normalized score, as seen by the increase in the mean normalized score from the left-hand-side of FIG. 6A (which corresponds to the monitoring without receiving O₂) to the right-hand-side of FIG. 6A (which corresponds to the monitoring while receiving O₂). The mean normalized score is indicated by a horizontal line L3 on the left-hand-side and by a horizontal line L4 on the right-hand-side. The normalized score as a function of time is indicated by a curve S1. As shown in FIG. 6B, on the second subject the opposite effect was observed. This is seen by the decrease in the mean normalized score from the left-hand-side of FIG. 6B (which corresponds to the monitoring without receiving O₂) to the right-hand-side of FIG. 6B (which corresponds to the monitoring while receiving O₂). The mean normalized score is indicated by a horizontal line L5 on the left-hand-side and by a horizontal line L6 on the right-hand-side. The normalized score as a function of time is indicated by a curve S2. Overall, the effect of O₂ administration was evaluated using a paired t-test and was found not to be significant (with a p-value of 0.058).

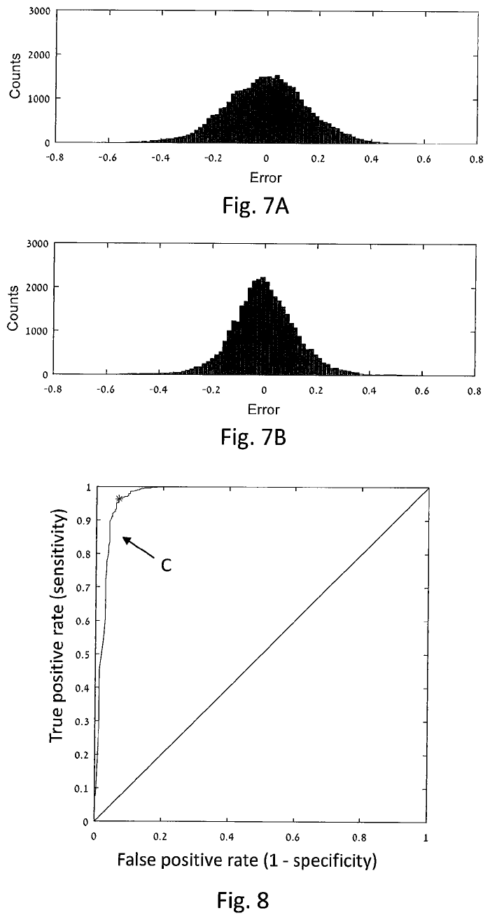

FIGS. 7A and 7B illustrate the effects of discounting and taking into account (by the ANN used in obtaining FIG. 4) demographic data (gender, age, and height) of the subjects, respectively. More specifically, the FIGS. 7A and 7B are statistical error curves. The points of FIG. 4 are classified according to their distances from the curve L1, thereby obtaining the respective errors associated therewith. The taking into account of the demographic data significantly improves the respiratory obstruction-level estimation, as can be seen from the curve of FIG. 7B being narrower and taller than the curve of FIG. 7A.

The performance of the auxiliary ANN (used in step 220, according to some embodiments) was also evaluated. Waveform features corresponding to the 8 waveform features of Table 1 were extracted from the raw capnograph signal for each segment corresponding to a single-breath. The waveform features of each single-breath signal were fed (as 8 distinct inputs) into an auxiliary ANN, which included one hidden layer with 5 nodes with a binary output classifying the single-breath signal as valid or invalid. To check the correctness of the classification, a reference data set was generated by having a specialist manually classify each of the single-breath signals, and a binary classification test was performed as detailed below.

FIG. 8 is a receiver operating characteristic (ROC) curve C obtained from the outputs of the auxiliary ANN and the reference data set. The accuracy was found to equal 0.96, the sensitivity 0.91, and the specificity 0.97, with the area under the curve C approximately equaling 0.98, demonstrating high-classification capability of the auxiliary ANN.

As used herein, the term “machine learning” encompasses also “deep learning”.

The Hjorth mobility M of a temporal function y(t) (e.g. a signal), which appears in Tables 2 and 3, is defined as M={square root over (var(\dot{y}(t))/var((t)))}, wherein \dot{y}(t) is the time derivative of y(t).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. A method of the disclosure may include a few of the steps described or all of the steps described. No particular step in a disclosed method is to be considered an essential step of that method, unless explicitly specified as such.

Although the disclosure is described in conjunction with specific embodiments thereof, it is evident that numerous alternatives, modifications and variations that are apparent to those skilled in the art may exist. Accordingly, the disclosure embraces all such alternatives, modifications and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

The phraseology and terminology employed herein are for descriptive purposes and should not be regarded as limiting. Citation or identification of any reference in this application shall not be construed as an admission that such reference is available as prior art to the disclosure. Section headings are used herein to ease understanding of the specification and should not be construed as necessarily limiting

What is claimed is:

1. A computer-implemented method for evaluating a respiratory obstruction-level in a subject with a respiratory condition, the method comprising:
obtaining a capnograph signal of a subject;
pre-processing the capnograph signal by
segmenting the capnograph signal into single-breath signals;
extracting a first plurality of waveform features from the capnograph signal;
based on the first plurality of waveform features, discarding from the capnograph signal invalid single-breath signal(s);
evaluating a respiratory obstruction-level of the subject by
extracting a second plurality of waveform features derived from the pre-processed signal;
feeding the second plurality of waveform features into a machine learning algorithm (MLA) to obtain a score quantifying the respiratory obstruction level of the subject.

2. The method of claim 1, wherein the respiratory condition is, or results from, asthma, chronic obstructive pulmonary disease (COPD), cystic fibrosis (CF), and/or a lung tumor(s).

3. The method of claim 1, wherein the MLA is an artificial neural network (ANN), a convolutional neural network, a random forest model, a fuzzy logic, a Bayesian network, a decision tree, a radial base function, a support vector machine, a linear regression model, a non-linear regression model, an expert system, or any combination thereof.

4. The method of claim 1, wherein inputs of the MLA comprise an input specifying the respiratory condition.

5. The method of claim 1, wherein the score allows distinguishing between at least 100 different respiratory obstruction levels.

6. The method of claim 1, wherein the score is substantially continuous.

7. The method of claim 1, wherein in said step of pre-processing the capnograph signal, the discarding of invalid single-breath signals is implemented using an auxiliary MLA.

8. The method of claim 1, wherein the second plurality of waveform features comprises multi-breath waveform features.

9. The method of claim 1, wherein the second plurality of waveform features comprises at least 20 waveform features.

10. The method of claim 1, wherein the second plurality of waveform features comprises the waveform features of Table 2 or any functions thereof.

11. The method of claim 1, wherein the second plurality of waveform features comprises at least 17 of the waveform features of Table 3 or any functions thereof.

12. The method of claim 1, wherein, in addition to the second plurality of waveform features, inputs of the MLA comprise demographic data characterizing the subject comprising at least one of gender, age, height, ethnicity, and/or weight of the subject.

13. The method of claim 1, wherein, in addition to the second plurality of waveform features, inputs of the MLA comprise treatment data comprising one or more of the following treatment parameters: a binary parameter specifying administration or no administration of O₂, rate of O₂ administration, and/or a binary parameter specifying provision or no provision of an inhaler.

14. The method of claim 1, wherein the first plurality of waveform features comprises at least 5 waveform features.

15. The method of claim 1, wherein the first plurality of waveform features comprises at least 5 of the waveform features of Table 1 or any functions thereof.

16. The method of claim 1, wherein the MLA is an ANN.

17. The method of claim 16, wherein weights and/or architecture of the ANN are dependent on the respiratory condition.

18. The method of claim 17, wherein the auxiliary MLA is an auxiliary ANN.

19. A computer-readable storage medium comprising software executable by a computer processor(s) for evaluating a respiratory obstruction-level in a subject with a respiratory condition, the software being configured, given a capnograph signal of a subject as an input, to implement steps of pre-processing the capnograph signal and evaluating a respiratory obstruction-level of the subject according to the method comprising:
obtaining a capnograph signal of a subject;
pre-processing the capnograph signal by
segmenting the capnograph signal into single-breath signals;
extracting a first plurality of waveform features from the capnograph signal;
based on the first plurality of waveform features, discarding from the capnograph signal invalid single-breath signal(s);
evaluating a respiratory obstruction-level of the subject by
extracting a second plurality of waveform features derived from the pre-processed signal;
feeding the second plurality of waveform features into a machine learning algorithm (MLA) to obtain a score quantifying the respiratory obstruction level of the subject.

20. A capnograph comprising the computer processer(s) and a computer-readable storage medium comprising software executable by a computer processor(s) for evaluating a respiratory obstruction-level in a subject with a respiratory condition, the software being configured, given a capnograph signal of a subject as an input, to implement steps of pre-processing the capnograph signal and evaluating a respiratory obstruction-level of the subject, the capnograph being thereby configured to implement a method, comprising:
obtaining a capnograph signal of a subject;
pre-processing the capnograph signal by
segmenting the capnograph signal into single-breath signals;
extracting a first plurality of waveform features from the capnograph signal;
based on the first plurality of waveform features, discarding from the capnograph signal invalid single-breath signal(s);
evaluating a respiratory obstruction-level of the subject by
extracting a second plurality of waveform features derived from the pre-processed signal;
feeding the second plurality of waveform features into a machine learning algorithm (MLA) to obtain a score quantifying the respiratory obstruction level of the subject.
